(19) United States
(12) Patent Application Publication
(10) Pub. No.: US 2007/0244501 A1
(43) Pub. Date: Oct. 18, 2007

(54) Medical balloons

(76) Inventors: Daniel J. Horn, Shoreview, MN (US); John Jianhua Chen, Plymouth, MN (US); John Blix, Maple Grove, MN (US); Jan Weber, Maple Grove, MN (US); Angela K. Volk, Rogers, MN (US); Richard C. Gunderson, Maple Grove, MN (US); Daniel K. Tomaschko, Savage, MN (US); Scott Schewe, Eden Prairie, MN (US)

Correspondence Address: MAYER & WILLIAMS PC, 251 NORTH AVENUE WEST, 2ND FLOOR, WESTFIELD, NJ, 07090, US

(21) Appl. No.: 11/406,022

(22) Filed: Apr. 18, 2006

(51) Int. Cl. A61M 29/00

(52) U.S. Cl. 606/194

(57) ABSTRACT

Described herein are medical balloons which contain one or more material regions that are configured to cause the balloons to preferentially fold into predetermined orientations upon deflation.

### FIELD OF THE INVENTION

The present invention relates to medical balloons, including those that are used in balloon angioplasty, among others.

### BACKGROUND OF THE INVENTION

Balloons mounted on the distal ends of catheters are widely used in medical treatment. A balloon may be used, for example, to widen a lumen (e.g., vessel) into which the catheter is inserted or to force open a blocked lumen. The requirements for the strength and size of the balloon vary widely depending on the balloon's intended use and the lumen size into which the catheter is inserted.

Perhaps the most demanding applications for such balloons are in balloon angioplasty (e.g., percutaneous transluminal coronary angioplasty or “PCTA”) in which catheters are inserted for long distances into extremely small vessels and are used to open stenoses of blood vessels by balloon inflation. These applications require thin-walled, high-strength balloons having predictable inflation properties. Thin walls are used, because the balloon's wall thickness limits the minimum diameter of the distal end of the catheter and therefore determines the ease of passage of the catheter through the vascular system and the limits on treatable vessel size. High strength is necessary because the balloon is used to push open stenoses, and the thin wall of the balloon must not burst under the high internal pressures necessary to accomplish this task (e.g., 10 to 25 atmospheres).

Subsequent to balloon inflation, the balloon is deflated and withdrawn from the vascular system, frequently in conjunction with a guide or sheath. After a balloon is inflated and deflated, there are multiple factors which may impact the force required to withdraw the balloon. One of the factors in minimizing this force is the ability of the balloon to deflate into an orientation which is amenable to balloon withdrawal.

### SUMMARY OF THE INVENTION

According to an aspect of the present invention, medical balloons are provided which contain one or more material regions that are configured to cause the balloons to preferentially fold into predetermined orientations upon deflation, for example, the profiles that the balloons had prior to inflation. Such orientations are typically adapted to allow the balloons to be readily withdrawn from the vasculature.

For example, in some embodiments, balloons are provided which contain one or more multilayer regions that are configured to cause the balloon to preferentially fold into a predetermined orientation upon deflation of the balloon. The multilayer regions may contain, for example, (a) one or more charged particle layers and (b) one or more charged polyelectrolyte layers.

In other embodiments of the invention, balloon catheters are provided which contain one or more elastomeric, heat-set and/or magnetic regions that are configured to cause the balloon to preferentially fold into a predetermined orientation upon deflation of the balloon.

These and other aspects, embodiments and advantages of the present invention will become immediately apparent to those of ordinary skill in the art upon reading the disclosure to follow.

### BRIEF DESCRIPTION OF THE FIGURES

FIGS. 1A-1C and 2A-2C are schematic axial cross-sections illustrating processes from forming balloons, in accordance with various embodiments of the invention.

FIGS. 3A-3B are schematic longitudinal cross-sections, illustrating a process for forming a balloon catheter, in accordance with an embodiment of the invention. FIGS. 4A-4B are schematic axial cross-sections, taken along the planes represented by lines a-a in FIGS. 3A-3B.

FIG. 4C is a schematic axial cross-section illustrating a process for forming the assembly of FIG. 4A, in accordance with an embodiment of the invention.

FIGS. 5A-5C are schematic axial cross-sections illustrating balloons, in accordance with various embodiments of the invention.

FIGS. 6A-6C are schematic longitudinal cross-sections, illustrating a process for forming a balloon catheter, in accordance with an embodiment of the invention. FIGS. 7A-7C are schematic axial cross-sections, taken along the planes represented by lines a-a in FIGS. 6A-6C.

FIGS. 8A-8F are schematic axial cross-sections illustrating molds for forming balloons, in accordance with various embodiments of the invention.

FIGS. 9A-9D are schematic longitudinal and axial cross-sections illustrating a process for forming a balloon catheter as well as its operation, in accordance with an embodiment of the invention.

FIG. 10A is a schematic longitudinal cross-section illustrating an inflated balloon catheter in accordance with an embodiment of the invention. FIG. 10B is a schematic axial cross-section taken along the plane represented by line a-a in FIG. 10A. FIG. 10C is a schematic axial cross-section illustrating the balloon catheter of FIGS. 10A-10B in a partially deflated state.

FIG. 11 is a schematic axial cross-section illustrating a partially inflated balloon catheter in accordance with an embodiment of the invention.

FIG. 12A is a schematic longitudinal cross-section illustrating an inflated balloon catheter in accordance with an embodiment of the invention. FIG. 12B is a schematic axial cross-section taken along the plane represented by line a-a in FIG. 12A. FIG. 12C is a schematic axial cross-section illustrating the balloon catheter of FIGS. 12A-12B in a partially deflated state.

FIG. 13A is a schematic longitudinal cross-section illustrating an inflated balloon catheter in accordance with an embodiment of the invention. FIG. 13B is a schematic axial cross-section taken along the plane represented by line a-a in FIG. 13A. FIG. 13C is a schematic axial cross-section illustrating the balloon catheter of FIGS. 13A-13B in a multi-wing configuration.

FIG. 14A is a schematic longitudinal cross-section illustrating an inflated balloon catheter in accordance with an embodiment of the invention. FIG. 14B is a schematic axial cross-section taken along the plane represented by line a-a in FIG. 14A. FIG. 14C is a schematic cross-section illustrating the balloon catheter of FIGS. 14A-14B in a partially deflated state.

FIGS. 15A is a schematic perspective illustration of an inflated balloon catheter in accordance with an embodiment of the invention. FIG. 15B is a schematic cross-sectional illustration of the balloon catheter of FIG. 15A, taken along line b-b. FIG. 15C is a schematic cross-section illustrating the balloon catheter of FIGS. 15A-15B in a partially deflated state.

FIG. 16 is a schematic illustration of a process of aligning the magnetic fields of ferromagnetic particles within a device in accordance with an embodiment of the invention.

### DETAILED DESCRIPTION OF THE INVENTION

As noted above, according to one aspect of the invention, balloons are provided which contain one or more material regions that are configured to cause the balloons to preferentially fold into predetermined orientations upon balloon deflation.

For example, in some embodiments, the balloons are provided with one or more multilayer regions that are configured to cause them to preferentially fold.

In some embodiments, the balloons are provided with one or more elastomeric, heat-set and/or magnetic regions that are configured for this purpose.

Each of these and other embodiments will be discussed below.

The balloons of the invention include balloons for insertion into and/or through a wide range of body lumens, including lumens selected, for example, from suitable members of the following: lumens of the cardiovascular system such as the heart, arteries (e.g., coronary, femoral, aorta, iliac, carotid and vertebro-basilar arteries) and veins, lumens of the genitourinary system such as the urethra (including prostatic urethra), bladder, ureters, vagina, uterus, spermatic and fallopian tubes, the nasolacrimal duct, the eustachian tube, lumens of the respiratory tract such as the trachea, bronchi, nasal passages and sinuses, lumens of the gastrointestinal tract such as the esophagus, gut, duodenum, small intestine, large intestine, rectum, biliary and pancreatic duct systems, lumens of the lymphatic system, the major body cavities (peritoneal, pleural, pericardial) and so forth.

Where the balloons are provided with one or more multilayer regions that are configured to cause preferential folding, in some embodiments, these regions preferably contain a plurality of alternating, oppositely charged layers, including the following: (a) one or more (typically a plurality of) charged particle layers, each containing charged particles, and (b) one or more (typically a plurality of) charged polyelectrolyte layers, each containing one or more charged polyelectrolyte species. The charges alternate between adjacent layers.

An advantage of such multilayer regions is that high strength characteristics may be achieved as deposited, as compared, for example, to certain other materials used to make balloons, where molecular orientation from a physical deformation process is required. Consequently, balloons may be built or reinforced in virtually any shape, without introducing mechanical weakness along the balloon body or significant variations in wall thickness.

Such multilayer regions may be applied using the so-called layer-by-layer technique in which a wide variety of substrates may be coated with charged materials via electrostatic self-assembly. In the layer-by-layer technique, a first layer having a first surface charge is typically deposited on an underlying substrate, followed by a second layer having a second surface charge that is opposite in sign to the surface charge of the first layer, and so forth. The charge on the outer layer is reversed upon deposition of each sequential layer. These techniques will be described in more detail below.

Substrates for the layer-by-layer technique include substrates that are incorporated into the finished device, as well as substrates that merely acts as templates for the layer-by-layer technique, but which are not found in the finished device (although a residue of the substrate may remain in certain embodiments).

In some embodiments, the one or more multilayer regions are applied to a substrate that corresponds to a folded or partially folded balloon or that reflects the shape of a folded or partially folded balloon.

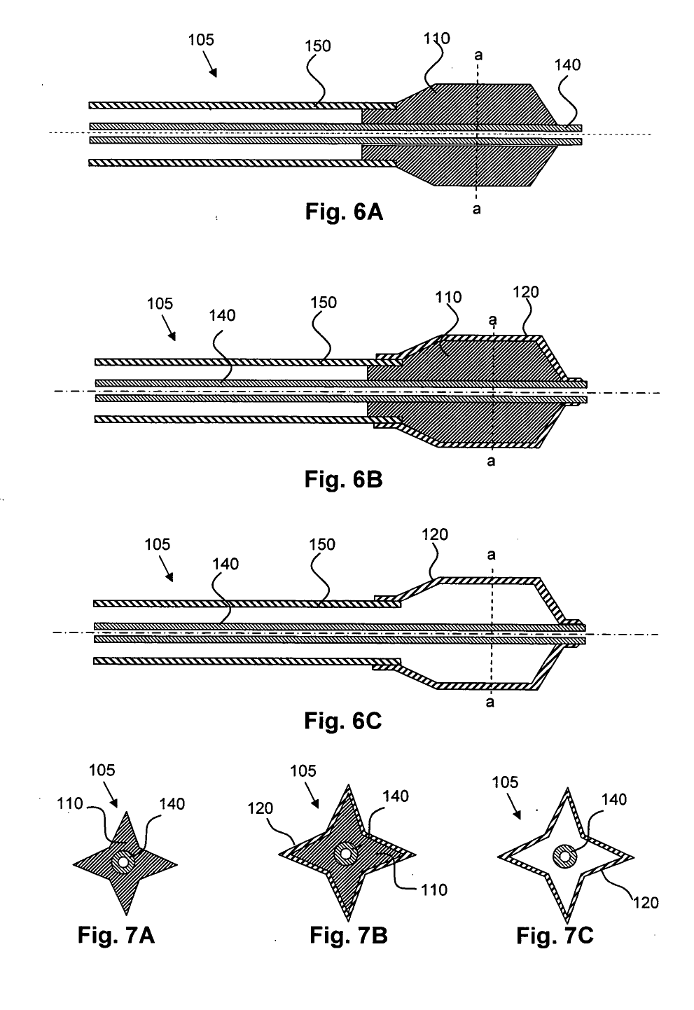

Turning now to FIGS. 6A-6C, an embodiment in which a balloon catheter in accordance with the invention is constructed will now be described. The embodiment described in these figures utilizes a removable mold for the formation of the multilayer region. The mold may be removed, for example, by melting, sublimation, combustion, dissolution or other process. Specific examples include materials that melt at relatively low temperatures, for instance, ice, as well as materials that melt at moderately elevated temperatures, for instance, dental waxes such as those available from MDL Dental Products, Inc., Seattle, Wash., USA. Other examples include materials that are essentially insoluble in cold water, but are soluble in hot water. Polyvinyl alcohol (PVOH) is one example of such a material. In other instances, however, balloons may be made using substrates that are not ultimately destroyed, several embodiments of which are discussed below.

Turning now to FIG. 6A, an assembly 105 is illustrated which includes a removable mold 110, an inner tubular member 140 forming a guidewire lumen, and an outer tubular member 150, which along with inner tubular member 140 defines an annular inflation lumen. A cross-section of the assembly 105 of FIG. 6A, taken along a plane corresponding to line a-a of FIG. 6A, is illustrated in FIG. 7A. The removable mold contains multiple protrusions (also referred to herein as wings or lobes) along at least a portion of its length.

Guidewire and inflation lumens are well known in the art and are commonly formed from materials such as nylons including nylon 12, thermoplastic polyester elastomers (e.g., Hytrel®), polyether-block co-polyamide polymers (e.g., poly(tetramethylene oxide)-b-polyamide-12 block copolymer, available from Elf Atochem as Pebax®), high density polyethylene, and polyurethane. Guidewire lumens are commonly provided with lubricious materials on their inner surfaces, for example, polytetrafluoroethylene or high density polyethylene.

In a next step, as illustrated in FIGS. 6B and 7B, a multilayer coating 120 is applied over the mold 110, for example, using layer-by-layer techniques such as those described in more detail below. In this embodiment, the multilayer coating 120 extends beyond the proximal (left) end of the mold 110, where it engages the outer tubular member 150, and extends beyond the distal (right) end of the mold 110, where it engages the inner tubular member 140. (Thus, in the present embodiment, the multilayer coating 120 is formed around the tubular members 140,150, although the multilayer coating can clearly be built independently of the tubular members and subsequently attached.)

Finally, the mold 110 is removed, thereby providing a finished balloon catheter having an inner tubular member 140, an outer tubular member 150, and a multilayer region 120 in the form of a balloon, as illustrated in FIGS. 6C and 7C.

When inflated, the multilayer balloon of FIGS. 6C and 7C is round. Due to the creases along the body of the multilayer balloon, which serve as hinge lines, the balloon folds back into a multiple-winged form when it is deflated.

In certain embodiments, the shape memory of the balloon 120 may be enhanced by crosslinking the multilayer region 120 (where the material is cross-linkable), for example, while it is still on the mold or while it is in a deflated state.

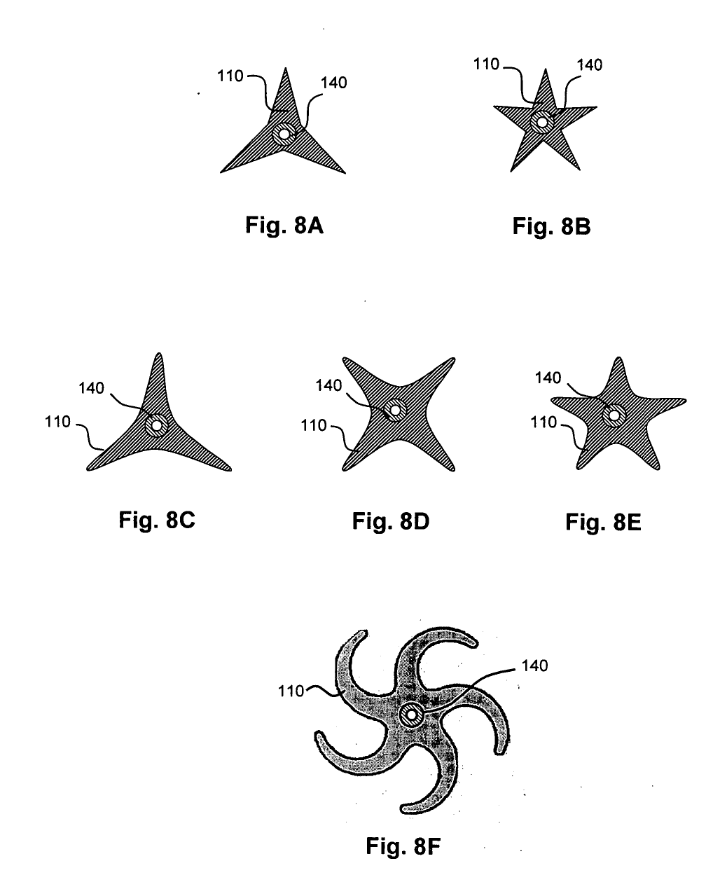

Of course innumerable variations on the above themes are possible. For example, rather than using a four-wing mold 110 as illustrated in FIG. 7A, one could employ a three-wing mold 110 as illustrated in FIG. 8A, a five-wing mold 110 as illustrated in FIG. 8B. Other multi-winged molds 110 are illustrated in FIGS. 8C, 8D, 8E and 8F. Of course, molds for two-winged, six-winged, seven-winged, eight-winged, etc. balloons are also encompassed by the present invention.

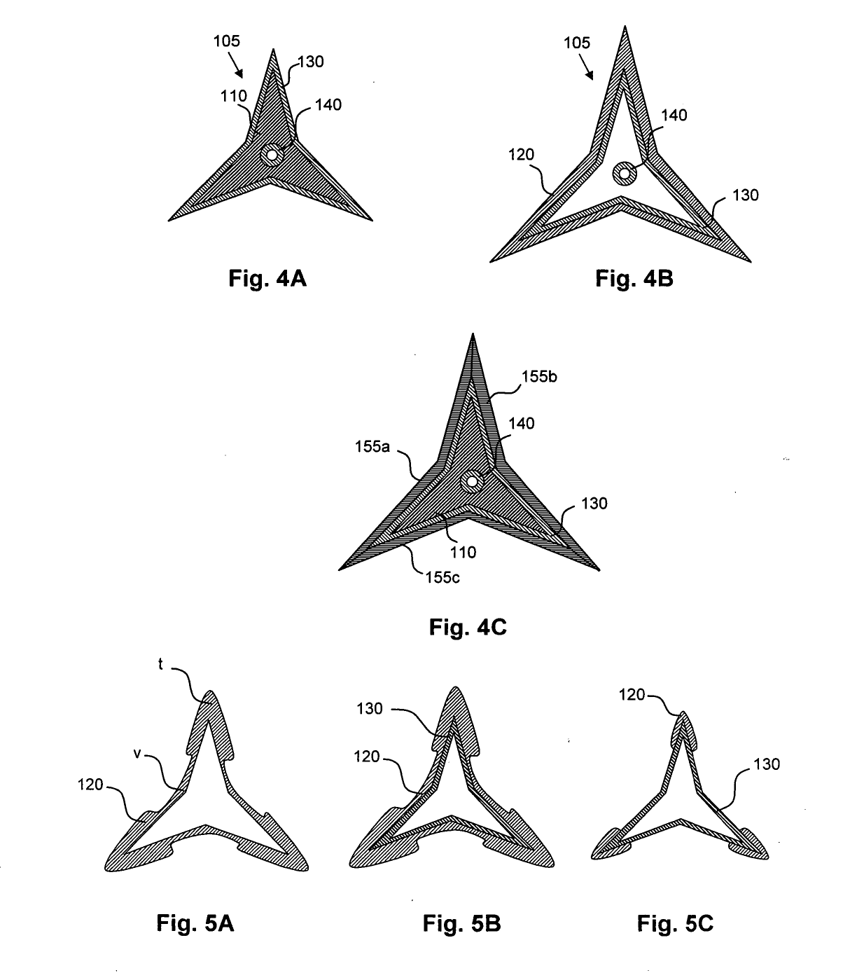

FIGS. 3A and 4A illustrate cross-sections similar to those of FIGS. 6A and 7A, except that (a) the removable material 110 has three wings rather than four and (b) a preformed balloon 130, such as one formed from Pebax® or another suitable material such as those listed above, is provided over the removable material 110. Such an assembly may be provided, for example, by filling the balloon 130 with a removable material 110 in liquid form (e.g., a low melting dental wax or other suitable material) while the balloon 130 is positioned within a mold (e.g., a three piece “clamshell” mold 155a, 155b, 155c, which may be formed from a polymeric, ceramic or metallic material) as illustrated in FIG. 4C. The balloon 130 is then removed from the mold after the material 110 has solidified to create the assembly of FIGS. 3A and 4A.

A multilayer coating 120 is then applied over the balloon 130 and removable material 110, in a fashion analogous to that described above in conjunction with FIGS. 6B and 7B, and the material 110 is then removed, likewise in a fashion analogous to that described above in conjunction with FIGS. 6C and 7C, thereby providing the structure illustrated in FIGS. 3B and 4B. As above, preferential hinge lines are created by this process, encouraging the balloon to fold back into a multiple-winged form when it is deflated.

In certain additional embodiments, it may be desirable to provide the multilayer region with additional layers, but only at certain locations, thereby creating regions of enhanced strength and stiffness in those locations. For instance, one may add additional layers to the multilayer region 120 in the vicinity of the wing-tips t, as illustrated in FIG. 5A or, conversely, in the vicinity of the valleys v between the wing tips t (not illustrated). This will increase the strength and stiffness of the multilayer region 120 at these positions as well as increasing the tendency the multilayer region 120 to refold at those positions upon deflation.

FIG. 5B is similar to FIG. 5A, except that a preformed balloon 130 is provided beneath the multilayer region 120. FIG. 5C is similar to FIG. 5B, except that multilayer regions 120 are only provided at the wing-tips of a preformed balloon 130.

In some embodiments, the material in the valleys may undergo axial compression during deflation. For example, the balloon may be initially blow molded in an original symmetrical round shape. After deflation, the wing tips are closer in configuration to the original shape than are the valleys, which undergo substantial axial compression. From an energetic standpoint, it makes sense to compress the weakest (e.g., thinnest) material. Thus, in such embodiments, configurations like that of FIGS. 5B and 5C may be desirable, as they provide a combination of weak valleys and strong tips.

In the above-described embodiments, the multilayer regions 120 are provided at the outer surface of the substrate (e.g., at the outer surface of a removable material 110 or at the outer surface of a preformed balloon 130). In other embodiments, on the other hand, multilayer regions may be provided at the inner surface of a substrate.

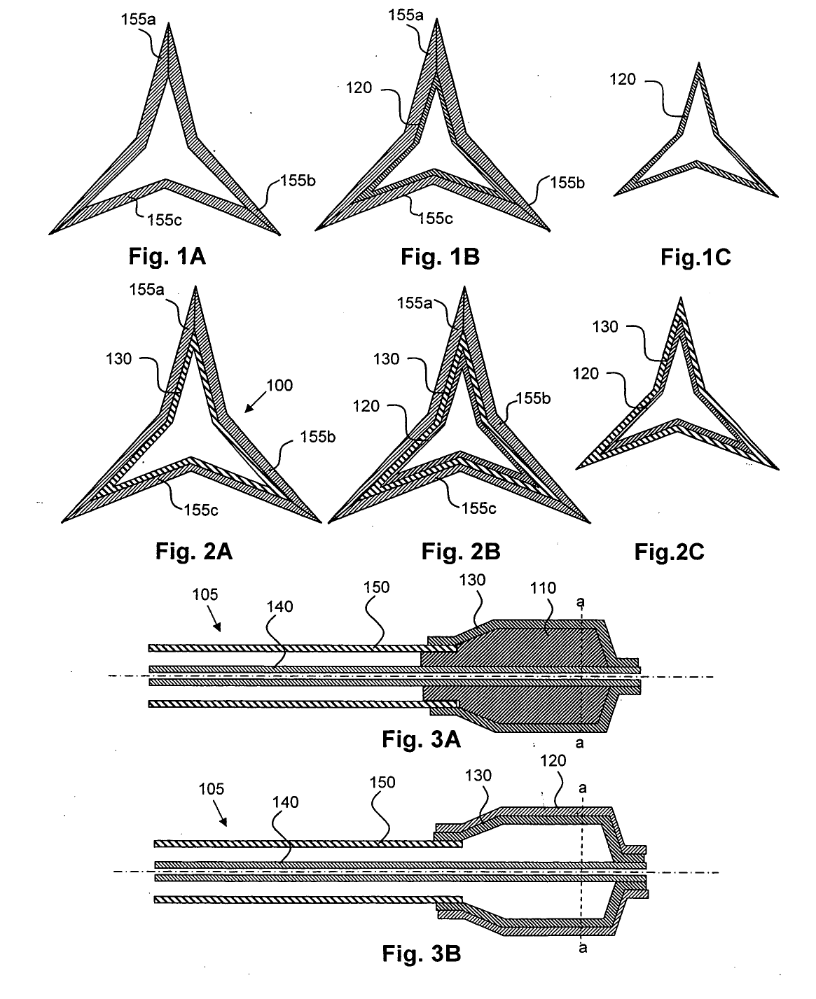

For example, turning now to FIG. 1A, a substrate (e.g., a three piece “clamshell” mold 155a, 155b, 155c) is illustrated schematically in cross-section, to the interior of which is applied a multilayer region 120 as shown in FIG. 1B. Removal of the mold 155a, 155b, 155c yields a balloon formed entirely from the multilayer region 120 as illustrated in FIG. 1C. This balloon may subsequently be attached (e.g., using a suitable adhesive) to inner and outer members, thereby providing a balloon catheter analogous to that illustrated in FIGS. 6C and 7C, if desired.

FIG. 2A illustrates an assembly 100 consisting of a preformed balloon 130 within a mold (e.g., a three piece “clamshell” type mold 155a, 155b, 155c). The assembly 100 may be created, for example, by expanding the 130 balloon within the mold 155a, 155b, 155c with a temporary adhesive on its outer surface and/or with a temporary adhesive applied to the inside surface of the mold 155a, 155b, 155c (e.g., a low melting point wax may be applied and heated prior to balloon expansion, then cooled afterward). Once the assembly 110 is created, a multilayer region 120 may be deposited on the inner surface of the balloon 130 as shown in FIG. 2B. Removal of the mold 155a, 155b, 155c (e.g., by heating the low melting wax) yields a substrate (i.e., balloon 130) with a multilayer region 120 disposed on its inner surface as illustrated in FIG. 2C.

Note that the balloon 130 may either become a permanent part of the device, or it may be removed. For example, the balloon may be a polymer balloon, such as a urethane balloon, which is subsequently dissolved in a solvent, such as tetrahydrofuran.

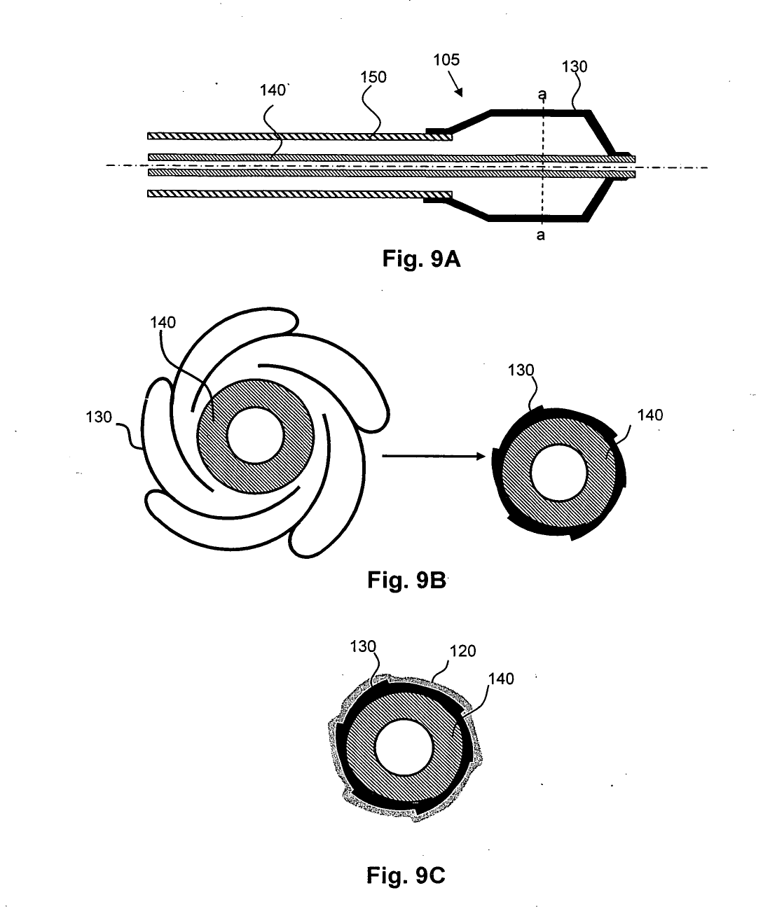

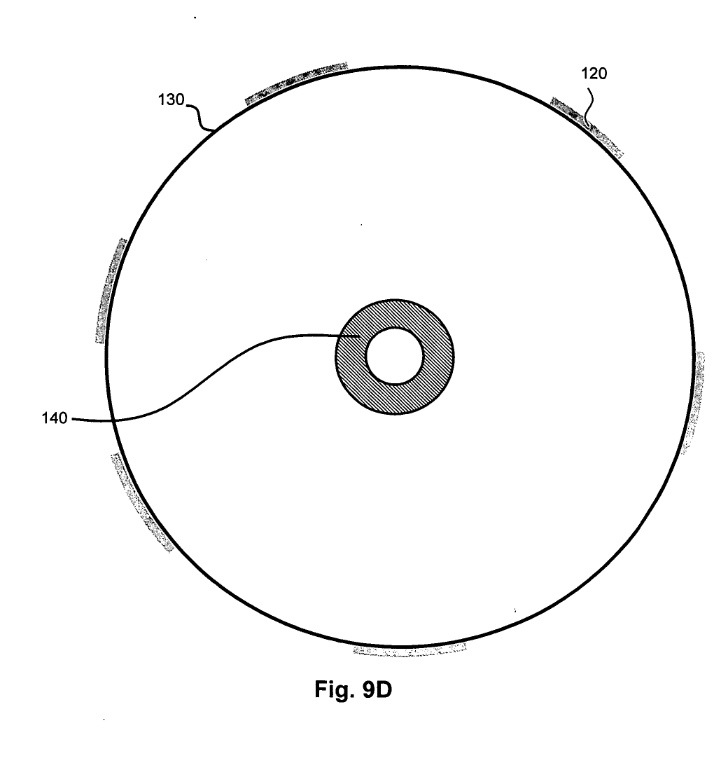

In yet another embodiment of the invention, a preformed balloon catheter 105, such as that shown inflated in longitudinal cross section in FIG. 9A, is provided. The balloon catheter 105 comprises an inner tubular member 140, an outer tubular member 150 and a multi-wing balloon 130. As shown in axial cross-section in FIG. 9B, the balloon 130 may be deflated and wrapped around the inner tubular member 140. Subsequently, a multilayer region 120 may be applied to the outer surface of the balloon 130 and the balloon is inflated as shown in FIG. 9C. The multilayer region 120 may be divided into discrete regions, for example, by cutting the multilayer region 120 into the discrete regions or by tearing of the multilayer region 120 into the discrete regions using the expansion force of the balloon. In other embodiments, a discrete multilayer region may be applied to the outer surface of the balloon. This discrete multilayer region can be formed by sliding a slotted tube (e.g., slotted in axial direction) over the balloon and attaching the individual axial elements to the balloon. The multilayer region is then deposited on the balloon in positions corresponding to the slots. The fully inflated balloon 130 with discrete multilayer regions 120 is shown in FIG. 9D. The balloon 130 is thereby configured to preferentially re-wrap upon deflation.

In other embodiments, a series of multilayer regions (e.g., in the form of longitudinal strips spaced at regular circumferential intervals) may also be applied to a round balloon (or applied to a round balloon mold, followed by balloon formation). As a result, the balloon will experience varying degrees of stiffness around its circumference. These variations in stiffness will cause differing responses to deflation, thereby achieving a preferential rewrap.

As indicated above, multilayer regions for preferential balloon rewrap in accordance with the present invention may be assembled using so-called layer-by-layer techniques. Layer-by-layer techniques may be used to coat a wide variety of substrate materials, including various balloon and mold materials, using charged materials via electrostatic self-assembly. In a typical layer-by-layer technique, multilayer growth proceeds through sequential steps, in which the substrate is alternately exposed to solutions of cationic and anionic species, frequently with intermittent rinsing between steps. In this way, a first layer having a first surface charge is typically deposited (or adsorbed) on the underlying substrate, followed by a second layer having a second surface charge that is opposite in sign to the surface charge of the first layer, and so forth. The charge on the outer layer is reversed upon deposition of each sequential layer.

Suitable substrate materials upon which the multilayer regions may be formed may be selected from a wide variety of materials, including (a) organic materials (e.g., materials containing 50 wt % or more organic species) and (b) inorganic materials (e.g., materials containing 50 wt % or more inorganic species), such as metallic materials (e.g., metals and metal alloys) and non-metallic inorganic materials (e.g., carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others).

Specific examples of organic materials include polymers (biostable or biodegradable) and other high molecular weight organic materials, which may be selected, for example, from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; various waxes, including low melting point waxes used for dental engineering (e.g., for so-called “lost wax” techniques); as well as blends and further copolymers of the above.

Certain substrate materials are inherently charged and thus readily lend themselves to layer-by-layer assembly techniques.

To the extent that the substrate material does not have an inherent net surface charge, a surface charge may nonetheless be provided. For example, where the substrate to be coated is conductive, a surface charge may be provided by applying an electrical potential to the same.

As another example, substrates, including polymeric substrates, may be chemically treated with various reagents, including reducing agents and oxidizing agents (e.g., sulfur trioxide for sulfonate formation), which modify their surfaces so as to provide them charged groups, for instance, amino, phosphate, sulfate, sulfonate, phosphonate and carboxylate groups, among many others.

Other techniques for providing surface charge include techniques whereby a surface region is treated with a reactive plasma. For example, gas discharge techniques have been used to functionalize polymer surfaces. Surface modification is obtained by exposing the surface to a partially ionized gas (i.e., to a plasma). Two types of processes are frequently described, depending on the operating pressure: corona discharge techniques (which are conducted at atmospheric pressure) and glow discharge techniques (which are conducted at reduced pressure). Because the plasma phase consists of a wide spectrum of reactive species (electrons, ions, etc.) these techniques have been used widely for functionalization of polymer surfaces.

Glow discharge techniques may be preferred over corona discharge techniques in certain embodiments, because the shape of the object to be treated is of minor importance during glow discharge processes. Moreover, glow discharge techniques are usually either operated in an etching or in a depositing mode, depending on the gas used, whereas corona discharge techniques are usually operated in an etching mode. A commonly employed glow discharge technique is radio-frequency glow discharge (RFGD).

Plasma treatment processes have been widely used to etch, crosslink and/or functionalize surfaces, with these processes occurring simultaneously at a surface that is exposed to a discharge of a non-polymerizable gas. The gas that is used primarily determines which of these processes is dominant. When gases like carbon monoxide (CO), carbon dioxide (CO₂), or oxygen (O₂) are used, functionalization with —COOH groups (which donate protons to form anionic groups) is commonly observed. When gases like ammonia, a propyl amine, or N₂/H₂ are employed, —NH₂ groups (which accept protons to form cationic groups) are commonly formed.

Functional group containing surfaces may also be obtained using plasma polymerization processes in which “monomers” are employed that contain functional groups. Allylamine (which produces —NH₂ groups) and acrylic acid (which produces —COOH groups) have been used for this purpose. By using a second feed gas (generally a non-polymerizable gas) in combination with the unsaturated monomer, it is possible to incorporate this second species in the plasma deposited layer. Examples of gas pairs include allylamine/NH₃ (which leads to enhanced production of —NH₂ groups) and acrylic acid/CO₂ (which leads to enhanced production of —COOH groups).

The above and further information on plasma processing may be found, for example, in “Functionalization of Polymer Surfaces,” Europlasma Technical Paper, May 8, 2004 and in U.S. Patent Application Publication No. 2003/0236323.

Laser processes may also be used to create surfaces having functionalized groups in any of a variety of patterns. A surface thus functionalized may then be used to create a patterned multilayer coating via layer-by-layer processes. The functionalization processes may be based, for example, on essentially the same principles as the plasma-based techniques of the preceding paragraphs. However, by using laser radiation such as UV laser processing (in conjunction with the gas or gases), one may create a localized plasma in the vicinity of the laser beam (e.g., just above the focal point of the beam), leading to localized surface functionalization.

As another example, the substrate can be provided with a positive charge by covalently linking species with functional groups having positive charge (e.g., amine, imine or other basic groups) or functional groups having a negative charge (e.g., carboxylic, phosphonic, phosphoric, sulfuric, sulfonic, or other acid groups) using covalent linkage methods well known in the art. Further information on covalent coupling may be found, for example, in U.S. Pub. No. 2005/0002865.

As another example, charged groups may be introduced by non-covalently binding charged compounds to the polymers, for example, based on van der Waals interactions, hydrogen bonding, hydrophilic/hydrophobic interactions and/or other interactions between the substrate and the charged compounds.

For instance, a surface charge may be provided on a substrate by exposing the substrate to a charged amphiphilic substance. Amphiphilic substances include any substance having hydrophilic and hydrophobic groups. Where used, the amphiphilic substance should have at least one electrically charged group to provide the substrate surface with a net electrical charge. Therefore, the amphiphilic substances that are used herein can also be referred to as an ionic amphiphilic substances. Amphiphilic polyelectrolytes are used as ionic amphiphilic substances in some embodiments.

In some embodiments, a surface charge is provided on a substrate by adsorbing polycations (for example, selected from polyethylenimine (PEI), protamine sulfate, polyallylamine, polydiallyldimethylammonium species, chitosan, gelatin, spermidine, and albumin, among others) or by adsorbing polyanions (for example, selected from polyacrylic acid, sodium alginate, polystyrene sulfonate (PSS), eudragit, gelatin, hyaluronic acid, carrageenan, chondroitin sulfate, and carboxymethylcellulose, among others) to the surface of the substrate as a first charged layer. PEI is commonly used for this purpose, as it strongly promotes adhesion to a variety of substrates. Although full coverage may not be obtained for the first layer, once several layers have been deposited, a full coverage should ultimately be obtained, and the influence of the substrate is expected to be negligible. The feasibility of this process has been demonstrated on glass substrates using charged polymeric (polyelectrolyte) materials. See, e.g., “Multilayer on solid planar substrates,” Multilayer thin films, sequential assembly of nanocomposite materials, Wiley-VCH ISBN 3-527-30440-1, Chapter 14; and “Surface-chemistry technology for microfluidics,” Hau, Winky L. W. et al. J. Micromech. Microeng. 13 (2003) 272-278.

Charge bearing species such as those above may be applied to the substrate by a variety of techniques. These techniques include, for example, full immersion techniques such as dipping techniques, spraying techniques, roll and brush coating techniques, ink jet techniques, spin coating techniques, web coating techniques and combinations of these processes, among others. Micro-polymer stamping may also be employed as described in S. Kidambi et al., “Selective Depositions on Polyelectrolyte Multilayers: Self-Assembled Monolayers of m-dPEG Acid as Molecular Templates” J. Am. Chem. Soc. 126, 4697-4703, 2004. The choice of the technique will depend on the requirements at hand. For example, full immersion techniques may be employed where it is desired to apply the species to an entire substrate, including surfaces that are hidden from view (e.g., surfaces which cannot be reached by line-of-sight techniques, such as spray techniques). On the other hand, spraying, roll coating, brush coating, ink jet printing, micropolymer stamping, etc. may be employed, for instance, where it is desired to apply the species only certain portions of the substrate (e.g., in the form of a pattern).

Once a substrate is provided with sufficient charge, it may be coated with a layer of an oppositely charged material. Multilayer regions are formed by repeated treatment with alternating, oppositely charged materials, i.e., by alternating treatment with materials that provide positive and negative surface charges. The layers self-assemble by means of electrostatic layer-by-layer deposition, thus forming a multilayered region over the substrate.

As noted above, the multilayer regions of the present invention typically include the following: (a) a plurality of charged particle layers, which contain one or more types of charged particles, and (b) a plurality of charged polyelectrolyte layers, which contain one or more types of charged polyelectrolytes.

As used herein, “polyelectrolytes” are polymers having multiple (e.g., 5 to 10 to 25 to 50 to 100 to 250 to 500 to 1000 or more) charged groups (e.g., ionically dissociable groups that provide cations and anions).

Frequently, the number of charged groups is so large that the polymers are soluble in polar solvents (including water) when in ionically dissociated form (also called polyions). Depending on the type of dissociable groups, polyelectrolytes may be classified as polyacids and polybases. When dissociated, polyacids form polyanions, with protons being split off. Examples of polyacids are polyphosphoric acids, polyvinylsulfuric acids, polyvinylsulfonic acids, polyvinylphosphonic acids and polyacrylic acids. Examples of the corresponding salts, which are also called polysalts, are polyphosphates, polyvinylsulfates, polyvinylsulfonates, polyvinylphosphonates and polyacrylates. Polybases contain groups which are capable of accepting protons, e.g., by reaction with acids, with a salt being formed. Examples of polybases having dissociable groups within their backbone and/or side groups are polyallylamine, polyethylimine, polyvinylamine and polyvinylpyridine. By accepting protons, polybases form polycations.

Some polyelectrolytes have both anionic and cationic groups, but nonetheless will have a net negative charge, for example, because the anionic groups outnumber the cationic groups, or will have a net positive charge, for example, because the cationic groups outnumber the anionic groups. In this regard, the net charge of a particular polyelectrolyte may change with the pH of its surrounding environment. Polyelectrolytes containing both cationic and anionic groups are generally categorized herein as either polycations or polyanions, depending on which groups predominate.

Thus, as defined herein, the term polyelectrolyte embraces a wide range of species, including polycations and their precursors (e.g., polybases, polysalts, etc.), polyanions and their precursors (e.g., polyacids, polysalts, etc.), polymers having multiple anionic and cationic groups (e.g., polymers having multiple acidic and basic groups such as a variety of proteins), ionomers (polyelectrolytes in which a small but significant proportion of the constitutional units carry charges), and so forth.

Linear or branched polyelectrolytes may be used in some embodiments. Using branched polyelectrolytes can lead to less compact polyelectrolyte multilayers having a higher degree of wall porosity.

Polyelectrolyte molecules may be crosslinked within or/and between the individual layers in some embodiments (e.g., by crosslinking amino groups with aldehydes, etc.) to increase stability and/or induce further stiffness in the polyelectrolyte multilayers, for example. As one specific example, charged polyelectrolyte layers (e.g., positively charged 2-nitro-N-methyl-4-diazonium-formaldehyde resin layers) may be photo-linked to adjacent charged particles (e.g., negatively charged polymethacrylic-acid-capped Fe₃O₄ nanoparticles) as described in Zhang et al., Thin Solid Films 429 (2003) 167-173.

Specific examples of suitable polycations may be selected, for instance, from the following: polyamines, including polyamidoamines, poly(amino methacrylates) including poly(dialkylaminoalkyl methacrylates) such as poly(dimethylaminoethyl methacrylate) and poly(diethylaminoethyl methacrylate), polyvinylamines, polyvinylpyridines including quaternary polyvinylpyridines such as poly(N-ethyl-4-vinylpyridine), poly(vinylbenzyltrimethylamines), polyallylamines such as poly(allylamine hydrochloride) (PAH) and poly(diallyldialklylamines) such as poly(diallyldimethylammonium chloride), spermine, spermidine, hexadimethrene bromide (polybrene), polyimines including polyalkyleneimines such as polyethyleneimines, polypropyleneimines and ethoxylated polyethyleneimines, basic peptides and proteins, including histone polypeptides and polymers containing lysine, arginine, ornithine and combinations thereof including poly-L-lysine, poly-D-lysine, poly-L,D-lysine, poly-L-arginine, poly-D-arginine, poly-D,L-arginine, poly-L-omithine, poly-D-ornithine, poly-L,D-omithine, gelatin, albumin, protamine and protamine sulfate, and polycationic polysaccharides such as cationic starch and chitosan, as well as copolymers, derivatives and combinations of the preceding, among various others.

Specific examples of suitable polyanions may be selected, for instance, from the following: polysulfonates such as polyvinylsulfonates, poly(styrenesulfonates) such as poly(sodium styrenesulfonate) (PSS), sulfonated poly(tetrafluoroethylene), sulfonated polymers such as those described in U.S. Pat. No. 5,840,387, including sulfonated styrene-ethylene/butylene-styrene triblock copolymers, sulfonated styrenic homopolymers and copolymer such as a sulfonated versions of the polystyrene-polyolefin copolymers described in U.S. Pat. No. 6,545,097 to Pinchuk et al., which polymers may be sulfonated, for example, using the processes described in U.S. Pat. Nos. 5,840,387 and 5,468,574, as well as sulfonated versions of various other homopolymers and copolymers, polysulfates such as polyvinylsulfates, sulfated and non-sulfated glycosaminoglycans as well as certain proteoglycans, for example, heparin, heparin sulfate, chondroitin sulfate, keratan sulfate, dermatan sulfate, polycarboxylates such as acrylic acid polymers and salts thereof (e.g., ammonium, potassium, sodium, etc.), for instance, those available from Atofina and Polysciences Inc., methacrylic acid polymers and salts thereof (e.g., EUDRAGIT, a methacrylic acid and ethyl acrylate copolymer), carboxymethylcellulose, carboxymethylamylose and carboxylic acid derivatives of various other polymers, polyanionic peptides and proteins such as glutamic acid polymers and copolymers, aspartic acid polymers and copolymers, polymers and copolymers of uronic acids such as mannuronic acid, galatcuronic acid and guluronic acid, and their salts, for example, alginic acid and sodium alginate, hyaluronic acid, gelatin, and carrageenan, polyphosphates such as phosphoric acid derivatives of various polymers, polyphosphonates such as polyvinylphosphonates, polysulfates such as polyvinylsulfates, as well as copolymers, derivatives and combinations of the preceding, among various others.

In some embodiments, non-polyelectrolyte, water-soluble polymers, for example, polyvinyl alcohol, among others, are provided with charge, for example, by adding acid to its water solution, thereby creating positive charges on the chains.

The particles for use in the charged particle-containing layers of the present invention can vary widely in size, but typically are nanoparticles that have at least one major dimension (e.g., the thickness for a nanoplates, the diameter for a nanospheres, nanocylinders and nanotubes, etc.) that is less than 1000 nm, more typically less than 100 nm. Hence, for example, nanoplates typically have at least one dimension (e.g., thickness) that is less than 1000 nm, other nanoparticles typically have at least two orthogonal dimensions (e.g., thickness and width for nano-ribbons, diameter for cylindrical and tubular nanoparticles, etc.) that are less than 1000 nm, while still other nanoparticles typically have three orthogonal dimensions that are less than 1000 nm (e.g., the diameter for nanospheres).

A wide variety of particles are available for use in the charged particle layers of the present invention including, for example, carbon, ceramic and metallic nanoparticles including nanoplates, nano-ribbons, nanotubes, and nanospheres, and other nanoparticles. Specific examples of nanoplates include synthetic or natural phyllosilicates including clays and micas (which may optionally be intercalated and/or exfoliated) such as montmorillonite, hectorite, hydrotalcite, vermiculite and laponite. Specific examples of nanotubes and nanofibers include single-wall, so-called “few-wall,” and multi-wall carbon nanotubes, vapor grown carbon fibers, alumina nanofibers, titanium oxide nanofibers, tungsten oxide nanofibers, tantalum oxide nanofibers, zirconium oxide nanofibers, and silicate nanofibers such as aluminum silicate nanofibers. Specific examples of further nanoparticles (e.g., nanoparticles having three orthogonal dimensions that are less than 1000 nm) include fullerenes (e.g., “Buckey balls”), silica nanoparticles, gold nanoparticles, aluminum oxide nanoparticles, titanium oxide nanoparticles, tungsten oxide nanoparticles, tantalum oxide nanoparticles, zirconium oxide nanoparticles, dendrimers, and monomeric silicates such as polyhedral oligomeric silsequioxanes (POSS), including various functionalized POSS and polymerized POSS.

With respect to functionalized gold nanoparticles, it is noted that these particles may help to create a radio-opaque layer. Gold nanoparticles may be made positively charged by applying an outer layer of lysine to the same. See, for example, “DNA-mediated electrostatic assembly of gold nanoparticles into linear arrays by a simple drop-coating procedure,” Murali Sastrya and Ashavani Kumar, Applied Physics Letters, Vol. 78, No. 19, 7 May 2001.

Other preferred groups of nanoparticles for the practice of the present invention are carbon nanofibers and single- and multi-wall carbon nanotubes that have a diameter ranging from 0.5 nm to 200 nm.

In this regard, carbon nanotubes, especially single-wall carbon nanotubes (SWNT), have remarkable mechanical properties, and may provide enhanced strength in composites, such as polymer composites. SWNT polymer composites are commonly prepared by polymer blending or by in situ polymerization techniques. Unfortunately, even with surface modification of the SWNT, phase separation is problematic due to the vastly different molecular mobilities of the components. To overcome phase separation issues between the SWNT and the polymer, layer-by-layer assembly has been used in which alternating layers of SWNT and polymeric material have been deposited. See Arif A. Mamedov et al., “Molecular design of strong single-wall carbon nanotube/polyelectrolyte multilayer composites,” Nature Material, Vol. 1, No. 3, 2002, pages 191-194, the entire disclosure of which is incorporated by reference.

The nature of particulate reinforced materials, such as multilayer materials containing carbon nanotubes, is that high strength characteristics may be achieved as deposited, as compared, for example, to various other materials used to make balloons, in which molecular orientation via a physical deformation process is employed. Consequently, balloons may be built in virtually any shape, without introducing mechanical weakness along the balloon body or significant variations in wall thickness, which preferentially revert to the as-molded configuration upon deflation.

As with substrates, various techniques are available for providing charges on nanoparticles that are not inherently charged. For example, a surface charge can be provided by adsorbing or otherwise attaching species on the nanoparticles which have a net positive or negative charge, for example, charged amphiphilic substance such as amphiphilic polyelectrolytes and cationic and anionic surfactants (see above). Moreover, where the nanoparticles are sufficiently stable, surface charges can sometimes be established by exposure to highly acidic conditions. For example, it is known that carbon nanoparticles, such as carbon nanotubes, can be partially oxidized by refluxing in strong acid to form carboxylic acid groups (which ionize to become negatively charged carboxyl groups) on the nanoparticles. Functionalized carbon nanotubes are also available commercially, for example, from Nanocyl S. A., B-5060 Sambreville BELGIUM, examples of which include carboxylated carbon nanotubes (Nanocyl®-2151) and —NH₂ functionalized carbon nanotubes (Nanocyl®-3152). Establishing a surface charge on nanoparticles is also advantageous in that a relatively stable and uniform suspension of the nanoparticles is commonly achieved, due at least in part to electrostatic stabilization effects.

In the present invention, layer-by-layer assembly is preferably conducted by exposing a selected charged substrate to solutions or suspensions that contain species of alternating net charge, for example, solutions or suspensions that contain charged nanoparticles, charged polyelectrolytes, or both. The concentration of the charged entities within these solutions and suspensions can vary widely, but will commonly be in the range of from 0.01 to 10 mg/ml.

The solutions and suspensions containing the charged species (e.g., solutions/suspensions of polyelectrolytes, charged nanoparticles, or both) may be applied to the charged substrate surface using a variety of techniques including those discussed above, for example, dipping techniques, spraying techniques, roll and brush coating techniques, spin coating techniques, web coating techniques, ink jet techniques, microstamping techniques, and combinations of these processes. As a specific example, layers can be applied over an underlying substrate by immersing the entire substrate into a solution or suspension containing the charged species, or by immersing half of the substrate into the solution or suspension, flipping the same, and immersing the other half of the substrate into the solution or suspension to complete the coating. In some embodiments, the substrate is rinsed after application of each charged species layer.

Using these and other techniques, multiple layers of alternating charge may be applied over an underlying substrate, including the application of one or more (typically a plurality of) charged nanoparticle layers and the application of one or more (typically a plurality of) charged polyelectrolyte layers. For example, in some embodiments, between 10 and 2000, more typically between 30 and 500 layers are applied over the substrate. The total thickness of the multilayer region that is assembled will typically range, for example, from 10 nanometers to 40 micrometers (microns), more typically, for example, between 100 nanometers and 10 microns.

In certain embodiments, the multilayer region comprises an alternating series of negatively charged nanoparticle layers and positively charged polyelectrolyte layers. In certain other embodiments, the multilayer region comprises an alternating series of positively charged nanoparticle layers and negatively charged polyelectrolyte layers. In certain other embodiments, more than one charged polyelectrolyte layer will be provided in succession. In certain other embodiments, more than one charged particle layer will be provided in succession. Clearly, innumerable variations are possible.

One preferred material for use in forming charged polyelectrolyte layers in accordance with the present invention is polyethyleneimine (PEI). PEI is an amphiphilic polyelectrolyte and thus is useful for establishing initial charged layers on substrates and can be used to provide subsequent polyelectrolyte layers as well. Being positively charged, PEI is usefull in combination with adjacent layers that contain negatively charged species, for example, carboxyl functionalized carbon nanotubes, among others. PEI having a molecular weight of about 70,000 is available from Sigma Aldrich. For example, to form a multilayer stack, the substrate can be dipped in a solution of PEI, rinsed, dipped in a suspension of carbon nanotubes, and so forth, with the number of alternating layers established ultimately depending, for example, upon the desired thickness and strength of the final multilayer region.

The PEI layer can also be followed by a layer of a negatively charged polyelectrolyte such as polyacrylic acid (PAA). The negatively charged polyelectrolyte is useful, for instance, in combination with adjacent layers that contain positively charged species, such as positively charged nanoparticles, for example dendrimers and functionalized gold nanoparticles, or positively charged polyelectrolytes such as PEI (e.g., where it is desired to establish multiple polyelectrolyte layers beneath, between and/or above the nanoparticle layers).

A variety of outer top layers can be provided for the multilayer regions of the present invention. For instance, the outer top layer may be a charged nanoparticle layer, a charged polyelectrolyte layer, and so forth. As a specific example, the outer top layer may be a carbon nanoparticle layer (e.g., a layer of charged carbon nanotubes, C60 “Buckey balls”, etc.).

In other embodiments, an outer polymer layer is provided over the multilayer region (e.g., using conventional thermoplastic or solvent processing techniques), for example, to protect the outer surface of the multilayer region and to contain any debris in the unlikely event that the multilayer region becomes damaged (e.g., in the unlikely event of a balloon burst). Such polymer layers can be selected from the various polymeric materials listed above for use in connection with substrates.

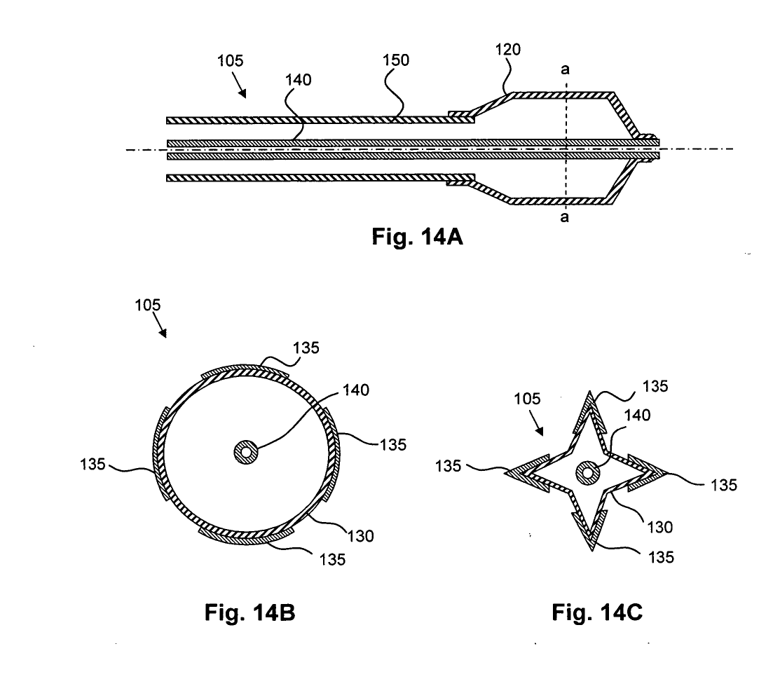

In additional embodiments of the invention, ceramic regions or polymer-ceramic composite regions of enhanced strength and stiffness are provided on balloon surfaces. Such regions may be provided, for example, at the tips of the folded wings, analogous to FIG. 5C. In this regard and with reference to FIGS. 14A-14C, a balloon catheter 105 is shown which comprises an inner tubular member 140, an outer tubular member 150, a balloon 130, and ceramic or hybrid polymer-ceramic regions 135 of enhanced strength and stiffness. The regions 135 may be found in the vicinity of the wing-tips, as illustrated in FIG. 14C. Similarly, regions 135 may be provided on a balloon surface in a fashion analogous to FIGS. 9A to 9D if desired.

As elsewhere herein, other configurations including other multi-winged configurations (e.g., 3-wing, 6-wing, 7-wing, etc.) may be employed by varying the number of regions 135. Moreover, although the ceramic or hybrid polymer-ceramic regions 135 run longitudinally along the length of the balloon, other configurations may be employed including, for example, helical configurations, among others.

Ceramic regions may be formed, for example, using sol-gel processing. In a typical sol-gel process, precursor materials, typically selected from inorganic metallic and semi-metallic salts, metallic and semi-metallic complexes/chelates, metallic and semi-metallic hydroxides, and organometallic and organo-semi-metallic compounds such as metal alkoxides and alkoxysilanes, are subjected to hydrolysis and condensation (also referred to sometimes as polymerization) reactions, thereby forming a “sol” (i.e., a suspension of solid particles within a liquid).

For example, an alkoxide of choice (such as a methoxide, ethoxide, isopropoxide, tert-butoxide, etc.) of a semi-metal or metal of choice (such as silicon, germanium aluminum, zirconium, titanium, tin, iron, hafnium, tantalum, molybdenum, tungsten, rhenium, iridium, etc.) may be dissolved in a suitable solvent, for example, in one or more alcohols. Subsequently, water or another aqueous solution, such as an acidic or basic aqueous solution (which aqueous solution can further contain organic solvent species such as alcohols) is added, causing hydrolysis and condensation to occur. If desired, additional agents can be added, such as agents to control the viscosity and/or surface tension of the sol.

Further processing of the sol enables solid materials to be made in a variety of different forms. For instance, coatings can be produced on a balloon substrate by spray coating, coating with an applicator (e.g., by roller or brush), ink-jet printing, screen printing, and so forth, of the sol onto the substrate, whereby a “wet gel” is formed. The wet gel is then dried. If the solvent in the wet gel is removed under supercritical conditions, a material commonly called an “aerogel” is obtained. If the gel is dried via freeze drying (lyophilization), the resulting material is commonly referred to as a “cryogel.” Drying at ambient temperature and ambient pressure leads to what is commonly referred to as a “xerogel.” Other drying possibilities are available including elevated temperature drying (e.g., in an oven), vacuum drying (e.g., at ambient or elevated temperatures), and so forth. Further information concerning sol-gel materials can be found, for example, in Viitala R. et al., “Surface properties of in vitro bioactive and non-bioactive sol-gel derived materials,” Biomaterials, 2002 August; 23(15):3073-86. Polymer-ceramic composite (hybrid) regions may be formed based on analogous processes, as well as principles of polymer synthesis, manipulation, processing, and so forth. Sol gel processes are suitable for use in conjunction with polymers and their precursors, for example, because they can be performed at ambient temperatures. A detailed review of various techniques for generating polymeric-ceramic composites can be found, for example, in G. Kickelbick, “Concepts for the incorporation of inorganic building blocks into organic polymers on a nanoscale” Prog. Polym. Sci., 28 (2003) 83-114.

It is known, for example, to impregnate a gel such as a xerogel with monomer and polymerize the monomer within the gel. Best results are obtained where non-covalent interactions between the monomer/polymer and the gel are sufficiently strong to prevent macroscopic phase separation.

Conversely, it is also known, for example, to generate polymeric-ceramic composites by conducting sol gel processing in the presence of a preformed polymer, which techniques can be successful, for example, where the polymer is soluble in the sol-forming solution (e.g., a solution containing alkoxy species, such as one containing tetraethyloxysilane (TEOS) or tetramethyloxysilane (TMOS)) and/or where the polymer has substantial non-covalent interactions with the ceramic phase (e.g., due to hydrogen bonding between hydroxyl groups and electronegative atoms within the polymeric and ceramic phases), which prevent macroscopic phase separation.

One way of improving the interactions between the polymeric and ceramic components is to employ a charged polymer, or ionomer. For this purpose, polymers may be functionalized with anionic groups, such as sulfonate or carboxylate groups, among others, or cationic groups, such as ammonium groups, among others.

Nanoscale phase domains may also be achieved by providing covalent interactions between the polymeric and ceramic phases. This result can be achieved via a number of known techniques, including the following: (a) providing species with both polymer and ceramic precursor groups and thereafter conducting polymerization and hydrolysis/condensation simultaneously, (b) providing a ceramic sol with polymer precursor groups (e.g., groups that are capable of participation in a polymerization reaction, such as vinyl groups or cyclic ether groups) and thereafter conducting an organic polymerization step, (c) providing polymers with ceramic precursor groups (e.g., groups that are capable of participation in hydrolysis/condensation, such as metal or semi-metal alkoxide groups), followed by hydrolysis/condensation of the precursor groups.

With respect to simultaneously conducting organic polymerization and hydrolysis/condensation reactions, various hybrid species are known which contain groups that can readily participate in each of these reactions. These hybrid species typically contain organic groups, such as vinyl-containing (—C═C), vinylidene-containing (>C═C), cyclic ether containing (e.g., 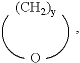
where y is 1 to 5) and/or siloxane-containing groups, which are capable of participating organic polymerization (typically in conjunction with a comonomer). These hybrid species also typically contain additional groups, such as -M(OR)ₘ groups (where M is a metal or semi-metal, m is an integer whose value will depend on the valency of M, typically ranging from 3 to 6, and the various R groups, which may be the same or different, are linear, branched or cyclic alkyl groups, aromatic groups or alky-aromatic groups of 1 to 10 carbon atoms, and preferably linear or branched alkyl groups having from 1 to 6 carbons, e.g., methyl, ethyl, propyl, isopropyl, and so forth), which are capable of participating in the hydrolysis/condensation reactions that are associated with sol-gel processing (typically in conjunction with another organometallic or organo-semi-metallic compound, such as M(OR)ₘ₊₁, where M, m, and R are defined above.) Specific known examples of such hybrid species include styrylethyltrimethoxysilane, 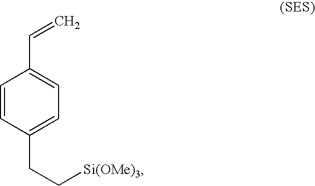
3-methacryloyloxypropyltrimethoxysilane 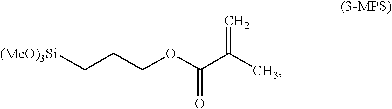
and glycidoxypropyltrimethoxysiliane 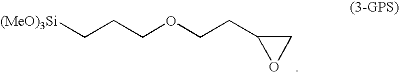
As indicated above, such hybrid species may be combined, for example, with (a) one or more optional organic monomers, for instance, vinyl-group-containing monomers (e.g., styrene, among many others), vinylidene-group-containing monomers (e.g., an alkyl methacrylate, where the alkyl portion may be R, as previously defined), or cyclic ether monomers (e.g., 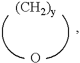
where y is 1 to 5), (b) one or more optional organometallic or organo-semi-metallic compounds, for instance, Si(OR)₄ where R is previously defined (e.g., TEOS or TMOS), (c) water, (d) a suitable catalysts, if required, and (e) energy (e.g., heat or photons), if required, at which time organic polymerization and hydrolysis/condensation commences. As a specific example, it is known to form composite materials having polymeric and ceramic phases from a mixture of 3-MPS, methyl methacrylate, TEOS, water, acid, and benzyol peroxide.

Hybrid species such as those described in the prior paragraph can also be used to form composite regions in accordance with routes (b) and (c) described above. For instance, in some cases, such hybrid species are first used to provide a ceramic phase (which contains the organic polymer precursor groups found in the hybrid species) followed by organic polymerization, typically in the presence of one or more comonomers. For example, a hybrid species containing one or more polymerizable organic groups, such as a vinyl, vinylidene, cyclic ether or siloxane groups, and one or more inorganic groups, such as -M(OR)ₘ groups (e.g., 3-MPS, SES or 3-GPS, among others) may be combined with a metallorganic compound such as a compound of the formula M(OR)ₘ₊₁ (e.g., TEOS or TMOS) in the presence of water and an acid catalyst such that hydrolysis and condensation take place. As a result, ceramic phases may be formed which have a range of groups that are capable of participation in polymerization reactions with a range of comonomers, including vinyl-, vinylidene-, cyclic-ether- and siloxane-containing monomers, via a range of organic polymerization reactions, including thermal, photochemical, anionic, cationic and radical polymerization methods, such as azobis(isobutyronitrile)- or peroxide-initiated polymerizations and controlled/“living” radical polymerizations, for instance, metal-catalyzed atom transfer radical polymerization (ATRP), stable free-radical polymerization (SFRP), nitroxide-mediated processes (NMP), and degenerative transfer (e.g., reversible addition-fragmentation chain transfer (RAFT)) processes, among others. These methods are well-detailed in the literature and are described, for example, in an article by Pyun and Matyjaszewski, “Synthesis of Nanocomposite Organic/Inorganic Hybrid Materials Using Controlled/“Living” Radical Polymerization,” Chem. Mater., 13:3436-3448 (2001), the contents of which are incorporated by reference in its entirety.

Conversely, in accordance with an aspect of route (c) above, polymers may be provided with inorganic groups that are capable of participation in hydrolysis/condensation, thereby becoming intimately associated with the ceramic phase. In these embodiments, hybrid species such as those discussed above may be employed in organic polymerization reactions via suitable polymerization techniques such as those listed above, typically in the presence of one or more comonomers. The inorganic groups incorporated into the resulting polymer are then available to participate in hydrolysis/condensation, e.g., using techniques such as those discussed above, thereby forming a ceramic phase that is covalently linked to the polymeric phase.
As an example, terephthalic acid, 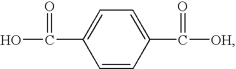
and an ethylene glycol/ceramic precursor, HO-M(OH)—O—(CH₂₎₂—OH may be polymerized to form a terephthalate copolymer of the structure, 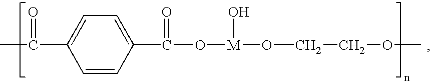
where n is an integer of 2 or more, typically 10 or more, 25 or more, 50 or more, 100 or more, 250 or more, 500 or more, or even 1000 or more. Ethylene glycol, HO—(CH₂₎₂—OH, or another glycol such as propylene glycol or tetramethylene glycol, may be included in the polymerization mixture in order to vary the ratio of the inorganic and organic units within the resulting hybrid polymer.

In other processes, preexisting polymers are provided with inorganic groups that are capable of participating in hydrolysis/condensation. For example, using appropriate linking chemistry, a wide variety of polymers, including polymers selected from those listed above, can be provided with groups for participation in sol-gel processing. A specific example of a group of polymers that are readily modified with organometallic or organo-semi-metallic groups for participation in sol gel processing are polymers having hydroxyl groups, including polyethers and polyesters. These polymers are sometimes referred to as polyols.

Numerous techniques are thus available for providing polymer-ceramic composite regions on substrates, (e.g., balloons or balloon precursors, such as parisons) in accordance with the present invention. For example, various techniques described above involve hydrolysis and condensation, which leads to the formation of a suspension containing a ceramic phase, which is analogous to the “sol” that is formed in sol-gel processing. This suspension also includes a polymer phase in several techniques. Such a sol may be applied to a substrate surface. Subsequent removal of water (as well as any other solvent species that may be present), results in the formation of a solid phase, which is analogous to the “gel” in sol-gel processing. As another example, where a polymer is present which has thermoplastic characteristics, the composite material may be heated to form a melt for further processing. Useful techniques for applying sols or melts on substrates include spray coating, coating with an applicator (e.g., by roller or brush), ink-jet printing, screen printing, extrusion, and so forth.

As indicated above, in additional embodiments of the invention, multilayer balloons are provided in which at least one layer of the balloon body is a heat-settable material. The heat-settable material layer provides the ability to heat set the balloon into a preferential configuration (e.g., a cylindrical balloon may be heat set into a winged shape, among others). Additional layers may be provided to balance the strength and flexibility properties of the balloon. For example, while some materials may maintain their heat-set across the entire working range of pressures of the balloon, in other materials, heat set may be lost once a certain inflation pressure or critical diameter is reached. In these instances, one or more layers of non-compliant material may be provided in the balloon structure, which limits the compliance of all the layers in the balloon. For example, a non-complaint material may be selected that displays an elongation at yield strength of 10% or less. As another example, a material may be selected that results in a balloon that displays less than 5% diameter growth within the pressure usage range.

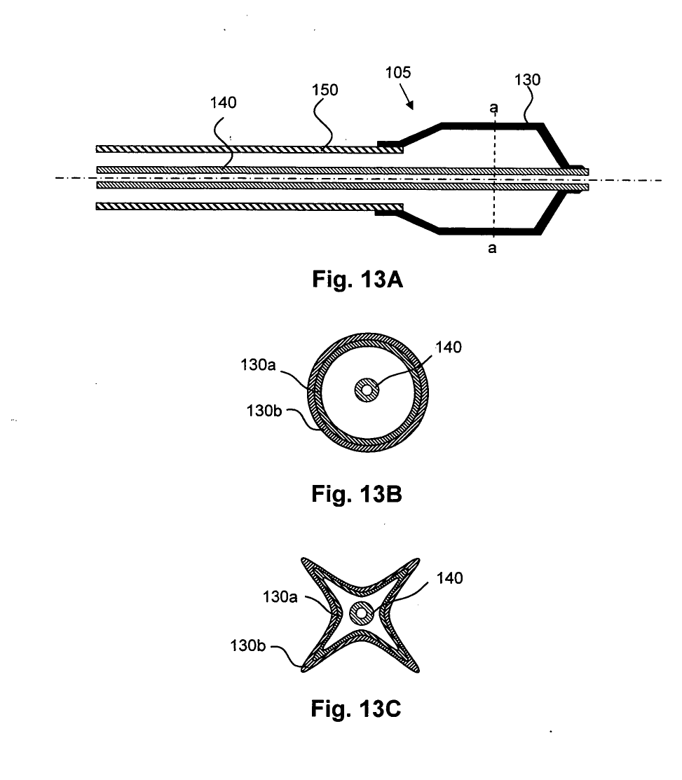

In one embodiment, a preformed balloon catheter 105, such as that shown inflated in longitudinal cross section in FIG. 13A, is provided. The balloon catheter 105 comprises an inner tubular member 140, an outer tubular member 150 and a balloon 130. As shown in axial cross-section in FIG. 13B, the balloon 130 of FIG. 13A is of a two-layer or “tube-in-tube” balloon design. Specifically, the balloon includes an outer layer 130b, which is formed from a low-compliance material such as aromatic polyester, aromatic polyamide or aromatic polyurethane, and an inner layer 130a, which is formed from a heat-settable material, for example, a semicrystalline polymer or a shape memory polymer, for instance, a polyamide polymer or copolymer such as nylon 12 or polyether-block-polyamide copolymers (e.g., Pebax®) or a shape memory polymer having a glass transition temperature (Tg) of about 35° C. During the balloon manufacture process, the balloon is shaped into a desired configuration, for example, into a multi-wing configuration like that shown in FIG. 13C. Such a configuration may be achieved, for example, by inflating the balloon in a multi-wing mold, among other techniques. While in the multi-wing configuration, the balloon may be heat set by heating the balloon. For example, for shape memory polymers, the polymer may be heated to temperatures above the Tg . In the case of semi-crystalline polymers such as polyamides, the polymer may be heated to temperatures sufficient to increase the crystallinity of the polymer (e.g., to temperatures above the Tg). Consequently, the balloon is urged into a multi-wing configuration upon deflation.

During use, after the balloon is inflated, the presence of the non-compliant layer 130b in the balloon construction reduces the balloon compliance and ensures that the “set” of the heat set material is kept, even after inflation to high pressures.

Although a four-wing configuration is illustrated, as discussed elsewhere herein, other configurations including other multi-winged configurations (e.g., 3-wing, 5-wing, 6-wing, etc.) may be employed. Moreover, the order of non-compliant layer and heat settable layer is interchangeable, among other variations

In further embodiments of the invention, multilayer balloons are provided in which at least one region is formed from an elastomeric material and at least one region is formed from a non-compliant material. Examples of non-compliant materials are described above. The elastomeric material induces the balloon to return to its pre-inflation state. Moreover, the elastomeric material is generally sufficiently elastic to avoid significantly affecting the fully deployed diameter of the balloon. Examples of elastomeric material for this purpose include, for example, low durometer grade polyurethane, among other materials.

The elastomeric material may be applied, for example, by adhering elastomeric material (e.g., in the form of strips or fibers) onto a balloon or onto material that is ultimately formed into a balloon (e.g., a tube parison). For example, strips may be attached at the distal and proximal balloon ends, for instance, using adhesives or thermal energy. The elastomeric material may also be applied, for example, in the form of a polymer solution that contains the elastomer using techniques including those discussed above, for example, dipping techniques, spraying techniques, roll and brush coating techniques, spin coating techniques, web coating techniques, ink jet techniques, microstamping techniques, and combinations of these processes.

In certain embodiments, the elastomeric material is applied only to discrete regions on the balloon. In certain embodiments, the elastomeric material covers the entire balloon. In certain embodiments, the elastomeric material is applied such that it covers the entire balloon and is subsequently selectively removed, for example, using processes such as laser ablation, selective chemical ablation, selective chemical bonding (e.g., photo-etching).

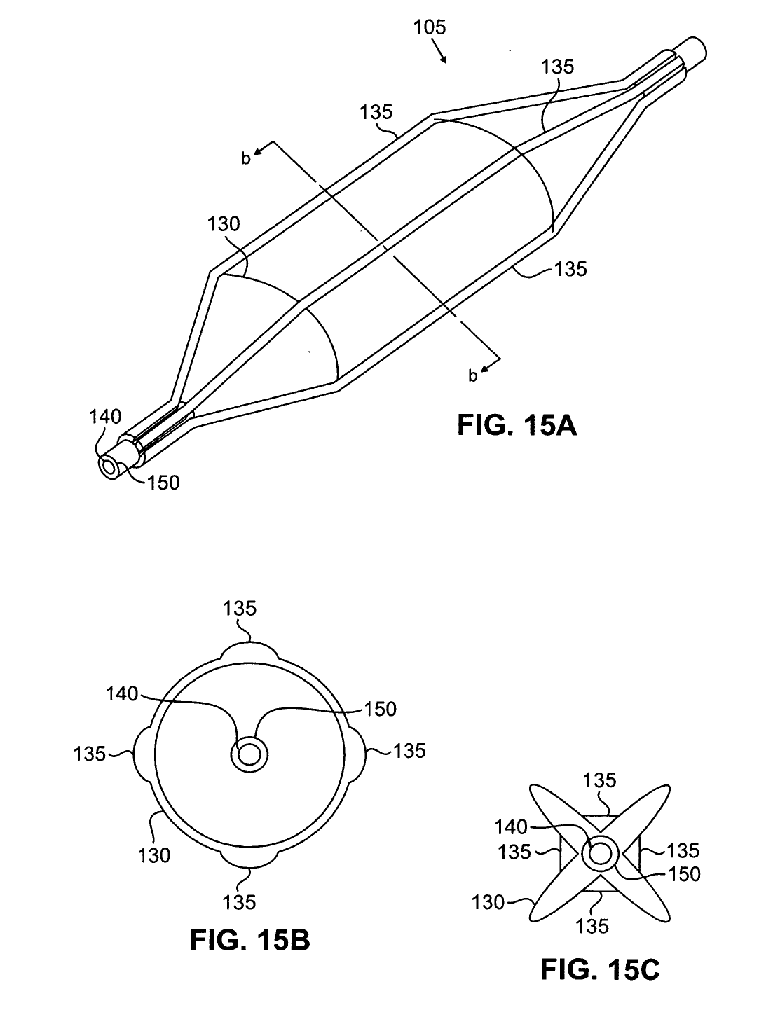

In one embodiment, a preformed balloon catheter 105 is provided, such as that shown inflated in perspective view in FIG. 15A and in cross-sectional view in FIG. 15B. The balloon catheter 105 comprises an inner tubular member 140, an outer tubular member 150 and a balloon 130. The balloon catheter further includes four elastomeric regions in the form of elastomeric strips 135 running along the length of the balloon 130. The balloon 130 may be formed, for example, from a low-compliance material such as those described above. Upon deflation, the elastomeric material 135 induces the balloon 130 to return to its pre-inflation state (which is in this instance a four-winged configuration as shown in FIG. 15C) by causing preferential folds along the length of the balloon 130. Although a four-wing configuration is illustrated, as discussed elsewhere herein, other configurations including other configurations may be employed, for example, other multi-winged configurations (e.g., 3-wing, 5-wing, 6-wing, etc.) may be employed by varying the number of elastomeric strips 135. Moreover, although the elastomeric strips 135 run longitudinally along the length of the balloon, other configurations may be employed including, for example, helical and circumferential configurations.

In still further embodiments of the invention, balloons are provided with one or more magnetic regions that are configured to cause the balloons to preferentially fold into predetermined orientations upon deflation.

These embodiments may employ, for example, two or more magnetic regions (e.g., because they contain magnetic materials, because that they contain electromagnets that become magnetic upon application of a current, etc.) or they may employ one or more magnetic regions and one or more regions that are susceptible to magnetic fields (e.g., because they contain paramagnetic materials such as iron).

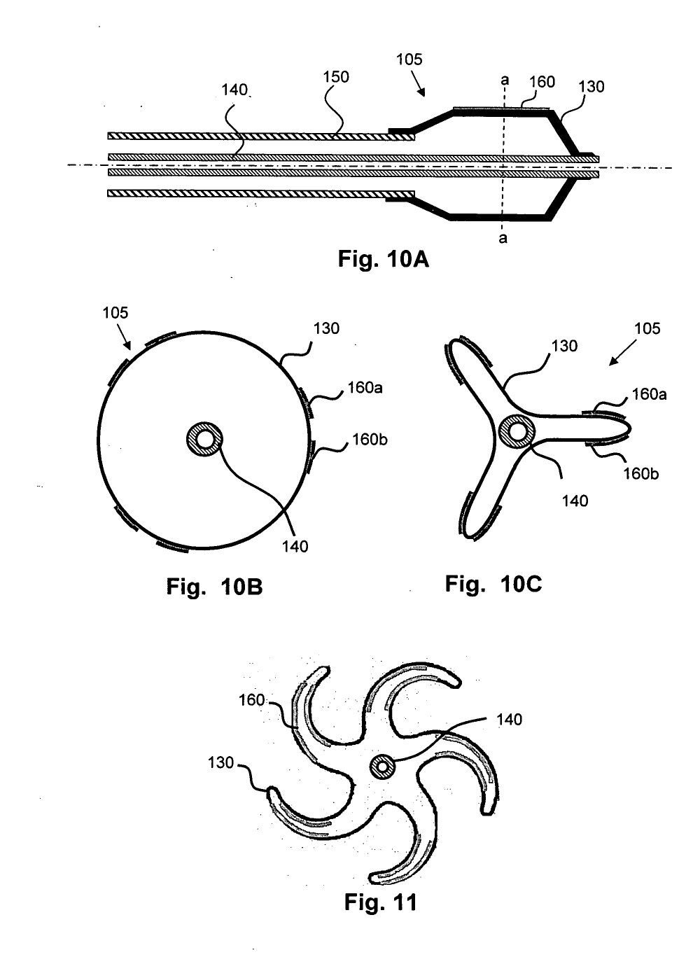

Turing now to FIGS. 10A and 10B, these are schematic longitudinal and axial cross-sections of a balloon catheter 105 having an inner tubular member 140, outer tubular member 150, an inflated balloon 130, and three pairs of strips 160a, 160b disposed on the outer surface of the balloon 130. For each pair of strips 160a, 160b, both may be magnetic, or one may be magnetic and the other paramagnetic, for example. FIG. 10C is a schematic axial cross-section of the catheter 105 of FIGS. 10A-B, but in a partially deflated state. Due to the placement of the magnetic strips 160 around the balloon and the magnetic interactions between them, upon balloon deflation, the pairs of strips 160a, 160b will prompt the balloon to collapse into a tri-winged profile like that shown in FIG. 10C.

As elsewhere herein, the invention is not restricted to three-winged designs. Moreover, while FIGS. 10B-C illustrate magnetic members applied to the outer surface of the balloon, it is also possible to apply magnetic members on the inner surface of the balloon as illustrated in the analogous five-winged cross-section of FIG. 11.

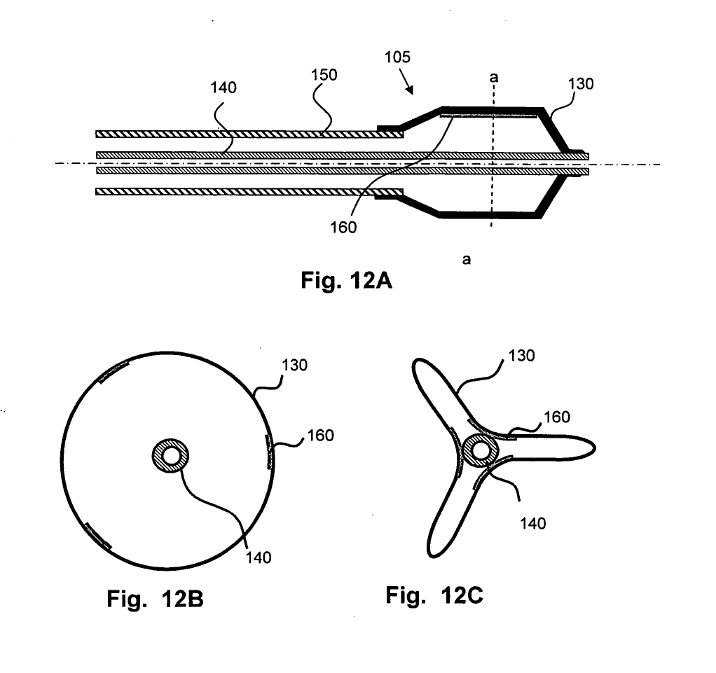

FIGS. 12A and 12B are schematic longitudinal and axial cross-sections of a balloon catheter 105 having an inner tubular member 140, which may be magnetic or paramagnetic, an outer tubular member 150, an inflated balloon 130, and three strips 160, which may be magnetic or paramagnetic, disposed on the inner surface of the balloon 130 at 120° intervals, or at some other irregular or regular (e.g., 90°, 72°, 60°, 51°, 45°, etc.) intervals. In a design like that of FIGS. 12A and 12B, for instance, (a) the tubular member 140 may be magnetic and the strips 160 may likewise be magnetic, (b) the tubular member 140 may be paramagnetic and the strips 160 may be magnetic, or (c) the strips 160 may be paramagnetic the tubular member 140 may be magnetic. Upon balloon deflation, due to the placement of the strips 106 around the balloon 130, the magnetic attraction between the strips 160 and the inner tubular member 140 will encourage the balloon 130 to collapse into a tri-winged form like that shown in FIG. 12C.

In certain embodiments, the tubular member 140 (or a guide wire within the tubular member 140) may be provided in the form of a permanent magnet or an externally activated electromagnet. The latter case allows, for example, an operator to control the refold of the balloon using a control mechanism located on the proximal hub or end of the catheter.

Ferromagnetic or paramagnetic regions, for instance ferromagnetic or paramagnetic layers in the form of strips such as those discussed in the preceding paragraphs, may be created, for example, by incorporating particles of ferromagnetic materials (e.g., particles of magnetite or other ferromagnetic materials) or paramagnetic materials (e.g., particles of iron or other paramagnetic materials) within the same.

Where the ferromagnetic or paramagnetic particles are charged, such regions may be formed, for example, using electrostatic layer-by-layer self-assembly techniques such as those described above.

As one example, gold coated cobalt particles larger than 10 nm are known to display ferromagnetic behavior. See, e.g., Y. Bao and K. M. Krishnan, “Preparation of functionalized and gold-coated cobalt nanocrystals for biomedical applications,” Journal of Magnetism and Magnetic Materials 293 (2005) 15-19. Cobalt creates a magnetic field that is three times stronger that that of similar magnetite particles. The gold coating is used to shield the non-biocompatible cobalt. As described in Z. Lu et al., “Magnetic Switch of Permeability for Polyelectrolyte Microcapsules Embedded with Co@Au Nanoparticles,” Langmuir, 21 (5), 2042-2050, 2005, such particles have a positive charge.

Other examples of charged ferromagnetic particles include magnetite particles and ferromagnetic polyoxometalates such as the polycationic polyoxometalates described in M. I. Khan et al., “Synthesis, structure and magnetic properties of a novel ferromagnetic cluster [FeV₆O₆{(OCH₂CH₂)2N(CH₂CH₂OH)}₆]Cl₂,” Inorganic Chemistry Communications-7 (2004) 54-57.

Thus, layers of gold-coated cobalt, charged magnetite or polycationic polyoxometalates may be used in layer-by-layer electrostatic self-assembly with essentially any combination of polycations and polyanions. (In this regard, Lu et al. supra only assembled 3-nanometer diameter particles, which are not ferromagnetic. However, utilizing the methodology from Bao and Krishnan supra, one may create larger particles beyond the 10 nm ferromagnetic threshold and utilize these in the layer-by-layer construction.)

Ferromagnetic and/or paramagnetic particles may be incorporated into polymeric regions in other ways. For instance, the particles may be dispersed within a polymer solution or polymer melt and applied to the inside and/or outside of a balloon (e.g., by dipping techniques, spraying, roll and brush coating, ink jet, stamping, spin coating, web coating, etc., masking the substrate as needed). As an example, R. Balasubramanian et al., “Dispersion and Stability Studies of Resorcinarene-Encapsulated Gold Nanoparticles,” Langmuir 2002, 18, 3676-3681, demonstrate the formation of stable dispersions of gold nanoparticles (and by extension, gold-plated cobalt nanoparticles) in toluene. By adding one or more polymers that can be dissolved in toluene (e.g., polystyrene and polystyrene-polyisobutylene block copolymers, among many others) a nanoparticle dispersion within a polymer solution may be obtained and applied to the inside and/or outside of a balloon using, for example, one of the above techniques.

As another example, a suspension of nanoparticles within a solvent may be applied to a balloon, which is formed from (or is coated with) a polymer that is swellable in that particular solvent. For example, a suspension of gold-plated cobalt nanoparticles in toluene as described in the prior paragraph may be applied to a balloon formed from (or coated with) a toluene-swellable polymer (e.g., a polyurethane such as Tecothane®), thereby embedding the nanoparticles in the polymer.

As yet another example, balloons are provided with one or more magnetic regions via sol gel processes. In certain of these embodiments, sol gel processing may be utilized to associate ferromagnetic and/or paramagnetic species with polymers (e.g., using techniques such as those as described above), for instance, within an interpenetrating network or at the molecular level as a true hybrid material which features covalent bonds between the metallic and polymeric components. Alkoxides that may be employed for this process include, for example, iron alkoxides such as iron(III) ethoxide or iron(II) isopropoxide, germanium alkoxides, and cobalt alkoxides. Further metallic species include mixed metal precursors such as nickel ferrite.

Moreover, device components, such as the inner tubular member described above, may also be rendered ferromagnetic or paramagnetic by including ferromagnetic or paramagnetic particles within the same. For example, where the component is formed from a polymer having thermoplastic characteristics, ferromagnetic or paramagnetic particles may be admixed with the polymer in the melt phase, and the resulting mixture processed by any of a variety of thermoplastic processing techniques, including compression molding, injection molding, blow molding, spinning, vacuum forming and calendaring, as well as extrusion into sheets, fibers, rods, tubes and other cross-sectional profiles of various lengths.

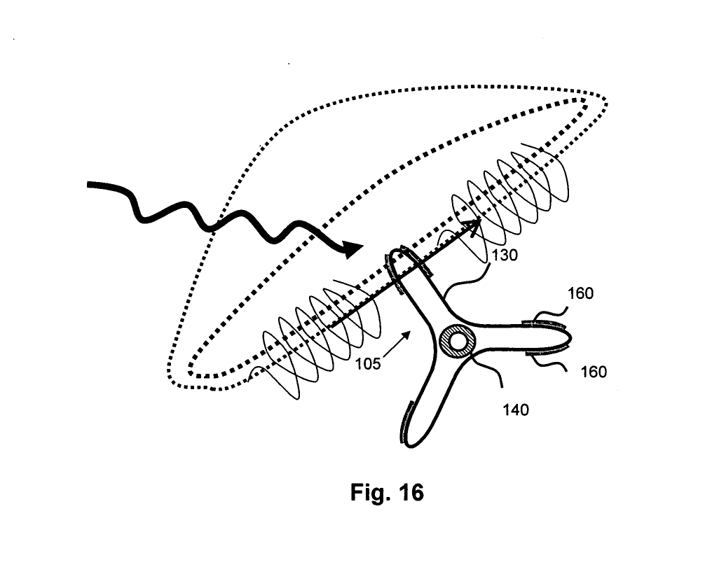

To reduce agglomeration and other issues, one may choose to make the particles ferromagnetic after the balloon assembly is formed. For example, to achieve proper ferromagnetic orientation, one may fold a balloon to the desired orientation upon deflation and apply a strong field to induce the proper ferromagnetic alignment. For example, referring now to FIG. 16A, folded wings of a balloon catheter 105, like that described in FIGS. 10A-10C above, may be inserted one by one into a gap between two coils. A strong magnetic field is induced (two field lines are represented by dashed lines) by sending a strong DC current through the coils, thereby orienting the ferromagnetic particles in the film. In the embodiment shown, the ferromagnetic particles are suspended in a photo-crosslinkable polymer suspension which is photo-crosslinked (as illustrated by the thick arrow) during or after magnetic alignment thereby freezing the orientation of the ferromagnetic particles in the polymer film.

Of course, device regions, including inner members and regions adjacent the balloon, may also be rendered magnetic using electromagnets.

### EXAMPLE 1

Polyvinyl alcohol (PVOH) series C-5 (purchased from Adept Polymers Limited, London) is insert molded at 190° C. to form a multi-winged balloon mold such as those discussed above (e.g., one having a cross section analogous to that of FIG. 8F in order to create a balloon in a partially rewrapped state). A metal core pin is embedded through the center of the mold.

The following solutions/suspensions are prepared: (1) Polyurethane Pellethane 70D (Dow Chemical, Midland, Mich.) in Tetrahydrofuran (THF) at a concentration of 5%; (2) polyethylenimine (PEI) (Aldrich) in water at a concentration of 1%; (3) polyacrylic acid (PAA) (Aldrich) in water at a concentration of 1%; and (4) carbon nanotubes (CNT) (Nanocyl®2151, Nanocyl S. A., BELGIUM) in water at a concentration of 0.6%.

A first layer of the polyurethane is deposited (by dipping) on the PVOH core. Then, a layer of PEI is deposited on top of the polyurethane layer. After this, 204 layers are deposited by repeating the following sequence seventeen times: PAA-PEI-CNT-PEI-CNT-PEI-CNT-PEI-CNT-PEI-CNT-PEI. The PAA layers are introduced to reinforce the electrostatic attraction.

After deposition of the layers, the metal core pin is pulled out of the mold and water at a temperature of 60° C. is flushed for 2 hours through the opening left by the core pin, thus dissolving the PVOH core.

### EXAMPLE 2

A heat-set balloon is formed using two tubes (a) and (b). Tube (a) is made of Vestamid L2101 F (nylon 12)(Degussa, Germany) and is employed as the balloon inner layer. Tube (b) is made of Isoplast 300 (polyurethane)(Dow Plastics, Midland, Mich., USA) and has an inside diameter that is 0.05 mm larger than the outside diameter of tube (a). Tube (a) is fed into tube (b) whereupon they are formed into a balloon in a standard balloon mold at 95° C. Then the formed balloon is inserted into a four-wing mold and heat set at 115° C.

### EXAMPLE 3

A nylon 12 (such as Vestamid L2101F) balloon formed at 140C is collapsed in a three wing arrangement and fixed in this shape by applying a vacuum to the interior of the balloon upon forming the wings and sealing the vacuum state by a valve. A PEI solution with a concentration of 5 mg/ml is prepared with distilled water containing 0.14 M NaCl. The three individual wings are each subsequently immersed up to 50% of the balloon height in PEI solution for 20 min, thus obtaining a precursor layer with a stable positive charge to initiate the LBL self-assembly process. PAH and PAA aqueous solutions (0.01 M based on molecular weights of the repeat units) are adjusted to the desired pH using 1 M HCl or 1 M NaOH. The PEI coated wings are immersed in the PAA solution for 10 minutes, followed by a rinsing step in water for 2 minutes, and the cycle is repeated for the PAH solution to built a PAH/PAA “bilayer”. PMAA capped Fe₃O₄ nanoparticles are prepared as explained in sections 2.1 and 2.2 of the article from Zhang above. After a first layer of PEI and 5 bilayers of PAH and PAA, PAA is replaced by PMAA capped Fe₃O₄ and a total of 20 additional bi-layers of PAH-PMAA are deposited. This is capped with again with 5 bilayers of PAA-PAH to avoid exposure of the nanoparticles directly to the outer surface. The coated balloons are dried by flushing with air at room temperature for 3 hours. Thermally induced cross linking at 130° C. for 3 hours converts a fraction of the ionic attachments between PAH and PAA to covalent amide bonds. The as formed balloons are magnetized by inserting the wings one by one in a gap within a solenoid and applying a 0.2 Tesla field for 2 minutes (e.g., 100 windings in a 10 cm solenoid, with a 5 mm diameter radius, having a core material on both sides of mumetal, relative permeability of 20,000, at a current of 10 mA). Polyethyleneimine (PEI) (Mw=75; 000) is available from Aldrich (Munich, Germany); PAH (MW=70 000) is available from Sigma-Aldrich (Milwaukee, Wis.); PAA (MW=90 000) is available from Polysciences (Warrington, Pa.).

Although various embodiments of the invention are specifically illustrated and described herein, it will be appreciated that modifications and variations of the present invention are covered by the above teachings without departing from the spirit and intended scope of the invention.

1. A medical balloon comprising a material region that is configured such that the balloon preferentially collapses into a predetermined orientation upon deflation of the balloon.

2. The medical balloon of claim 1, wherein said orientation matches the orientation of the balloon prior to inflation.

3. The medical balloon of claim 1, wherein said predetermined balloon orientation comprises three or more wings.

4. The medical balloon of claim 3, wherein upon deflation said wings wrap in the same circumferential direction around a longitudinal axis of said balloon.

5. The medical balloon of claim 3, wherein said material region is disposed substantially on a single side of said wings.

6. The medical balloon of claim 1, wherein said material region varies substantially in thickness.

7. The medical balloon of claim 6, wherein said material region is thicker at the tips of said wings.

8. The medical balloon of claim 6, wherein said material region is thicker in valleys between said wings.

9. The medical balloon of claim 1, wherein said material region is disposed over only a portion of an inside or outside surface of a polymeric balloon.

10. The medical balloon of claim 1, wherein said material region is disposed over at least a portion of an inside surface of a polymeric balloon.

11. The medical balloon of claim 10, wherein said material region is disposed over at least a portion of an outside surface of a polymeric balloon.

12. The medical balloon of claim 1, wherein said material region comprises (a) a plurality of layers comprising charged particles and (b) a plurality of layers comprising charged polyelectrolyte.

13. The medical balloon of claim 12, wherein said material region is formed on an inside or outside surface of a removable mold that corresponds in shape to a balloon that is in a partially collapsed configuration.

14. The medical balloon of clam 12, wherein said charged particle layers comprise nanoparticles selected from carbon nanoparticles, silicate nanoparticles, and ceramic nanoparticles.

15. The medical balloon of clam 12, wherein said charged particle layers comprise nanoparticles selected from carbon nanotubes, carbon nanofibers, fullerenes, ceramic nanotubes, ceramic nanofibers, phyllosilicates, monomeric silicates and dendrimers.

16. The medical balloon of clam 12, wherein said charged particle layers comprise single walled carbon nanotubes.

17. The medical balloon of clam 12, wherein said material region comprises a plurality of positively charged polyelectrolyte layers and a plurality of negatively charged polyelectrolyte layers.

18. The medical balloon of claim 12, wherein said material region comprises from 10 to 200 layers.

19. The medical balloon of claim 1, comprising a plurality of said material regions.

20. The medical balloon of claim 1, wherein said material region is formed on an inside of a balloon that is in a partially collapsed configuration.

21. The medical balloon of claim 1, wherein said material region is formed on an outside surface of a balloon that is in a partially collapsed configuration.

22. A balloon catheter comprising the medical balloon of claim 1 and an elongate member comprising an inflation lumen in fluid communication with an interior of said balloon.

23. The medical balloon of claim 1, wherein said material region is a magnetic region.

24. The medical balloon of claim 23, wherein said predetermined balloon orientation comprises three or more wings.

25. The medical balloon of claim 23, comprising a plurality of magnetic regions.

26. The medical balloon of claim 25, wherein said plurality of magnetic regions comprise a plurality of magnetic strips running longitudinally along at least a portion of the length of said balloon.

27. The medical balloon of claim 23, comprising a plurality of magnetic regions on an inside surface of said balloon.

28. The medical balloon of claim 23, comprising a plurality of magnetic regions on an outside surface of said balloon.

29. The medical balloon of claim 23, further comprising a paramagnetic region.

30. The medical balloon of claim 23, comprising a magnetic or paramagnetic inner member within the interior of said balloon and a plurality of magnetic or paramagnetic regions on an inside surface of said balloon, on an outside surface of said balloon, or within the wall of said balloon.

31. The medical balloon of claim 30, wherein said inner member is a guide wire or guide wire lumen that extends through said balloon.

32. The medical balloon of claim 23, comprising a magnetic inner member within said balloon and a plurality of magnetic or paramagnetic regions on an inside surface of said balloon, on an outside surface of said balloon, or within the wall of said balloon.

33. The medical balloon of claim 23, comprising a paramagnetic inner member within said balloon and a plurality of magnetic regions on an inside surface of said balloon, on an outside surface of said balloon or within the wall of said balloon.

34. The medical balloon of claim 23, wherein said magnetic region is a multi-layer region comprising (a) a plurality of layers comprising charged magnetic particles and (b) a plurality layers comprising charged polyelectrolyte.

35. The medical balloon of clam 34, wherein said charged magnetic particles comprise cobalt.

36. The medical balloon of claim 34, wherein said multilayer region comprises from 10 to 200 layers.

37. The medical balloon of claim 23, wherein said magnetic region comprises magnetic particles disposed within a polymer matrix.

38. The medical balloon of claim 23, wherein said magnetic region comprises a ceramic component.

39. The medical balloon of claim 23, wherein said magnetic region comprises polymeric and ceramic components.

40. The medical balloon of claim 39, wherein said ceramic component comprises a metal oxide selected from oxides of iron, germanium, cobalt and combinations thereof.

41. A balloon catheter comprising the medical balloon of claim 23 and an elongate member comprising an inflation lumen in fluid communication with an interior of said balloon.

42. The medical balloon of claim 1, wherein said material region comprises a heat-set material.

43. The medical balloon of claim 42, further comprising a non-compliant material.

44. The medical balloon of claim 43, wherein said heat-set material comprises a polymeric material selected from semi-crystalline polymers, shape memory polymers, and combinations thereof.

45. The medical balloon of claim 43, wherein said non-compliant material comprises a polymeric material selected from aromatic polyesters, aromatic polyamides and aromatic polyurethanes.

46. The medical balloon of claim 43, wherein said predetermined orientation is a multi-wing orientation that comprises three or more wings upon deflation.

47. The medical balloon of claim 46, wherein said balloon is heated, while in said multi-wing profile, to a temperature that is above a glass transition temperature of the heat-set material.

48. The medical balloon of claim 1, wherein said material region comprises a ceramic component.

49. The medical balloon of claim 48, further comprising a non-compliant material.

50. The medical balloon of claim 1, wherein said material region is a sol-gel layer.

51. The medical balloon of claim 1, wherein said material region comprises polymeric and ceramic components.

52. The medical balloon of claim 1, wherein said material region changes in material properties.

53. The medical balloon of claim 1, wherein said material region reduces the forces required to withdraw said balloon from a patient.
